(19) United States
(12) Patent Application Publication
(10) Pub. No.: US 2003/0161861 A1
(43) Pub. Date: Aug. 28, 2003

(54) Use of a cereal product for improving cognitive performance and mental well-being in a person, particularly in a child and an adolescent

(76) Inventors: Vincent Lang, Maisons-Alfort (FR); Magali Degouy, Viroflay (FR); Yann Champenois, Athis-Mons (FR)

Correspondence Address: FINNEGAN, HENDERSON, FARABOW, GARRETT & DUNNER, LLP, 1300 I STREET, NW, WASHINGTON, DC, 20005, US

(21) Appl. No.: 10/169,827

(22) PCT Filed: Jan. 9, 2001
(86) PCT No.: PCT/FR01/00055

(30) Foreign Application Priority Data
Jan. 10, 2000 (FR) 00/00303

(51) Int. Cl. A61K 47/00; A61K 31/718

(52) U.S. Cl. 424/439; 514/60

(57) ABSTRACT

The invention concerns a the use of a cereal product such as a biscuit or cracker having a slowly digestible starch content relative to the total starch content higher than about 12 wt %, preferably higher than about 20 wt %, to improve cognitive performances, in particular memory retention, attention, concentration, vigilance and/or mental well-being in people, and particularly in a child and an adolescent. Said cereal products can be eaten at breakfast.

The present invention relates to the use of cereal products having a slowly digestible starch content relative to the total starch content greater than 12%, preferably greater than 20%, to improve cognitive performance and/or mental well-being.

Learning is one of the essential components of human behavior because it allows lasting modifications thereto, thereby improving the functioning of the individual. Learning involves many functions such as the acquisition of knowledge, the memorization, release and analysis of information. The mechanisms of learning are still poorly known.

The brain is an organ which plays an essential role in learning, in relationship with all other organs of the individual. Research studies of the past few decades have shown that the supply of energy and the mineral and vitamin status of the individual have an influence on the expression and the accomplishment of their learning.

Our current societies have in common certain broad organizational traits. One of them, although not applicable in all societies worldwide, is learning in school. This is carried out during different periods of the day. For children, the morning period is often rich in diverse and varied learning activities. During the morning, a lot of effort in terms of attention, concentration, memorization and release of knowledge is demanded of children.

Such a demand is also valid for adults who also accomplish many physical and intellectual activities, at work or during their leisure activities.

It is for this reason that it is highly recommended to have breakfast in order to build up the energy reserves again after a night of fasting, and to supply the body with energy for its morning activities. This is particularly true for children, in whom energy renewal is very important.

In order to build up these energy reserves, a so-called “balanced” breakfast is generally proposed which is generally composed of four types of products:

a cereal product (bread, French toasts, Vienna-type products, breakfast cereals or biscuits),

a dairy product,

fruit or fruit juice

and a drink.

The balance of this type of breakfast is obtained by providing a suitable percentage and a sufficient quantity of lipids, carbohydrates and proteins. This type of supply effectively makes it possible to build up the reserves but does not necessarily bring about an improvement in the intellectual functions, in particular the cognitive functions as was stated earlier. Now, the research studies by the applicant have shown that certain forms of foods taken in particular at breakfast made it possible to improve the intellectual functions especially in children and adolescents.

The abstracts of CN 1 135288 and of CN 1 107655 mention biscuits containing various plant extracts which are thought to have beneficial effects on health, in particular by improving immunity, brain functions and vision.

Ross et al. (Am J Clin Nut, 1987) describe the glycemic and insulinemic indices of various cereal products; the lowest indices are observed, in the case of biscuits, for high fat contents.

Korol et al. (Am J Clin Nut, 1998) have pointed out that the level of circulating glucose influences certain cognitive functions, and may in particular improve memory disorders in elderly persons.

However, other studies have concluded that glucose did not have a role in these processes.

Unexpectedly, the applicants have shown that the regulation of the glycemic index, alone, was insufficient to increase these performances. The applicants have now demonstrated that certain cereal products significantly improve cognitive performance, by virtue of the choice of appropriate proportions between slowly digestible starch and the total starch present in the product. These products may have, moreover, moderate lipid levels.

In addition, the variation of the ratio between the slowly digestible starch and the total starch (which induces in parallel variations in the content of slowly available glucose relative to the total carbohydrate content) makes it possible to obtain cereal products having a lower glycemic index, at an otherwise equivalent composition, and in particular at an equivalent fat content. Thus, a product having a lipid level of 17 g/100 g and a slowly available glucose content>15% has a glycemic index of 45. A product having a lipid level of 9 g/100 g and a slowly available glucose content>15% has a glycemic index of 59. By contrast, a product having a lipid level of 12 g/100 g and a slowly available glucose content<7% has a glycemic index of 70.

Accordingly, the subject of the present invention is the use of cereal products, in particular of biscuits or crackers, having a slowly digestible starch content relative to the total starch content greater than about 12% by weight, preferably greater than about 20%, to improve the mental well-being and/or cognitive performance, in particular memorization, attention, concentration and/or vigilance in a person, and particularly in a child and an adolescent.

The present invention also relates to cereal products, in particular biscuits or crackers, for promoting attention, concentration, vigilance, memorization and/or mental well-being in a person, and in particular in a child and an adolescent, characterized in that they have a slowly digestible starch content relative to the total starch content greater than about 12% and preferably greater than about 20%.

The preferred starch content of the cereal products which can be used according to the invention is from 30 to 70 g per 100 g of dry matter, in particular from 34 to 60 g per 100 g of dry matter.

Preferably, the cereal products which can be used according to the invention have a slowly available glucose content relative to the total carbohydrate content greater than about 10%, still preferably greater than about 15%.

The preferred carbohydrate content of the cereal products which can be used according to the invention is from 60 to 90 g per 100 g of dry matter.

The sugar content of the cereal products which can be used according to the invention is preferably from 2 to 40 g per 100 g of dry matter. The sugar may be a monosaccharide and/or a disaccharide, and in particular glucose, sucrose, fructose and/or maltose. The moisture contents of the cereal products according to the invention may vary, and in particular may be of the order of 7 to 10% by weight. However, products which are particularly suitable for carrying out the invention comprise moisture levels of less than 5%, in particular of the order of 3 to 4%.

Another important characteristic of the cereal products which can be used according to the invention is their lipid content. Indeed, it is commonly accepted that a high level of lipids influences the rate of digestion of carbohydrates by slowing it down via an effect on gastric emptying. Now, it has been possible to demonstrate that moderate lipid levels nevertheless make it possible to obtain the desired improvements in the context of the present invention, which has the advantage of avoiding the accumulation of lipids. Thus, the lipid content of the cereal products which can be used according to the invention is preferably from 3 to 25 g per 100 g of dry matter, still preferably from 10 to 20 g per 100 g of dry matter and still more particularly from 14 to 20 g per 100 g of dry matter.

In particular, cereal products prepared according to the present invention and having lipid levels of less than 15 g per 100 g of dry matter, in particular of the order of 12 g per 100 g and balanced starch supplies relative to the carbohydrates make it possible to improve the various aspects of cognitive performance.

Surprisingly, the protein content of the cereal products which can be used according to the invention is low, preferably 5 to 11 g per 100 g of dry matter. Indeed, this is in contrast to a comparative study carried out in adults relating to the effects of protein-rich meals compared to carbohydrate-rich meals and recommending the use of proteins to improve attention (Spring et al., “effects of protein and carbohydrate meals on mood and performance: interactions with sex and ages”, J. Psychiat. Res, 1982, vol. 17, 2, 155-167).

Although the cereal products according to the invention allow improvement in cognitive performance and/or mental well-being in general, regardless of the time at which they are consumed, cognitive performance and/or mental well-being are more particularly improved when the cereal products which can be used according to the invention are consumed during breakfast.

In addition, the ingestion of cereal products containing contents of slowly digestible starch according to the invention makes it possible to maintain cognitive performance, and in particular learning and memorization capacities even when the body is subjected to conditions of depletion of energy reserves.

The present invention also relates to a nontherapeutic method for promoting attention, concentration, vigilance and/or memorization in a person, and in particular in a child and an adolescent, characterized in that it involves the consumption, preferably during breakfast, of cereal products, in particular of biscuits or crackers according to the invention.

The term “cereal product” in the present invention represents a preparation predominantly consisting of flour, fats, water and sweetening substances for sweetened products.

Starch is generally considered as being slowly digested. However, the rate and degree of starch digestion and absorption may vary considerably according to the source of starch and of the food technology which is applied thereto during the manufacture of the food.

The slowly digestible starch content of the cereal products which can be used according to the invention may therefore be due both to their starch source and also to the forming technology used for their manufacture. The content of slowly available glucose reflects the rate at which the glucose derived from sugar and starch becomes available for absorption in the human small intestine.

The slowly digestible starch content relative to the total starch content and the slowly available glucose content relative to the total carbohydrate content of the cereal products which can be used according to the present invention are measured by means of the Englyst method (Englyst H N, Veenstra J., Hudson G J., 1996, Measurement of rapidly available glucose (RAG) in plant foods: a potential in vitro predictor of the glycaemic response, British Journal of Nutrition, 75, 327-337 and Englyst K N., Englyst H N., Hudson G J., Cole T J., Cummings J H., 1999, Rapidly available glucose in foods: a measurement that reflects the glycemic response, Am J Clin Nutr, 69, 448-454). This method makes it possible to classify foods as a function of the in vitro bioavailability of their starch and of the digestibility of all the carbohydrates available. The classification of certain foods are presented in the following table 1.

TABLE 1
Bioavailability in vitro (Englyst method) of various foods

| | Slowly digestible | |
|---|---|---|
| | starch/total starch | Slowly available glucose/ |
| | (%) | total available carbohydrates (%) |
| Spaghetti | 42 | 74 |
| Kidney beans | 42 | 84 |
| Instant mashed | 1 | 8 |
| potatoes | | |
| Cornflakes | 3 | 3 |
| Breakfast | 2 | 1 |
| cereals for | | |
| children | | |
| Porridge | 9 | 10 |
| White bread | 9 | 15 |
| Wholemeal bread | 7 | 12 |
| Baguette | 0 | 0 |
| Petit beurre | 14 | 11 |
| biscuit | | |
| Breakfast | 38 | 23 |
| biscuit | | |

The cereal products which can be used according to the invention preferably have, in addition, a particularly low glycemic index, in particular of less than 60, preferably less than 50 and still more preferably less than 45.

The cereal products which can be used according to the invention are preferably obtained by forming technologies known to persons skilled in the art, such as laminated, laminated puff, and cut doughs, or by wire cut or rotary doughs.

Examples of processes used for obtaining the cereal products which can be used according to the invention are as follows:

1. Cereal Process for Sweetened Doughs

The predominant raw materials are flour, sweetening substances and fat. They are mixed with other ingredients such as water, salt, baking powder, and the like, in a kneader. This stage is called kneading. The consistency of this dough determines its passage over the manufacturing line.

If this dough is bound (network provided by proteins), and forms a dough block, after a variable rest period, it will be shaped by laminating cylinders so as to make a dough strip of 1 to 2 mm. It will then be cut, by a roto-slicer cylinder, to the desired biscuit shape and size. Laminated and cut doughs are therefore obtained.

If this dough has no cohesion and resembles sand, it will be molded to the desired shape and size of the biscuit and unmolded by a rotary machine. These are rotary doughs.

If this dough has no cohesion, it is sticky, it will be dressed in a wire cutting device which will cut dough pieces. These are wire cut doughs.

These doughs may then be glazed, and will then be baked in an oven. On leaving the oven, the biscuits will be cooled before they are packaged.

2. Cereal Process for Neutral or Salty Doughs

The predominant raw materials are flour, water, an active ingredient according to the processes (enzyme or yeast or leavening agent).

These ingredients are mixed, in part (fermented cracker) or as a whole. They are fermented for a variable period of 1 to 24 hours, at room temperature, or at high temperature according to the process. The dough is laminated, and optionally sheeted and then cut with a roto-slicer to the desired cracker size. The crackers are then baked, and optionally sprayed with fat and flavored and then cooled and packaged. These are laminated, or laminated puff, and cut doughs.

The invention will be illustrated by the examples which follow, which relate to studies during which components of cognitive performance were tested on an animal model, which can be extrapolated to people.

In example 1, reference is made to the following figures:

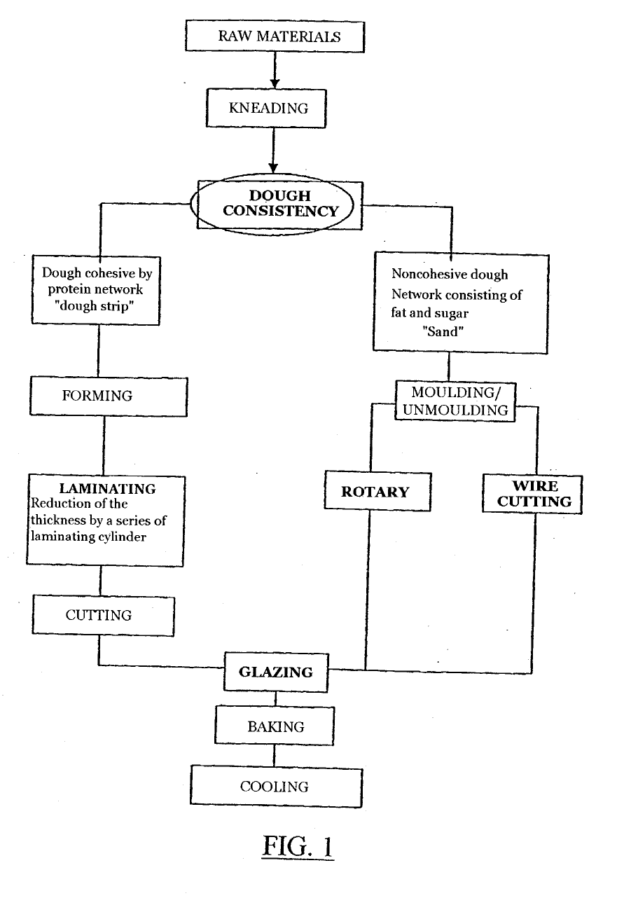

FIG. 1 represents various types of processes for manufacturing cereal products according to the invention.

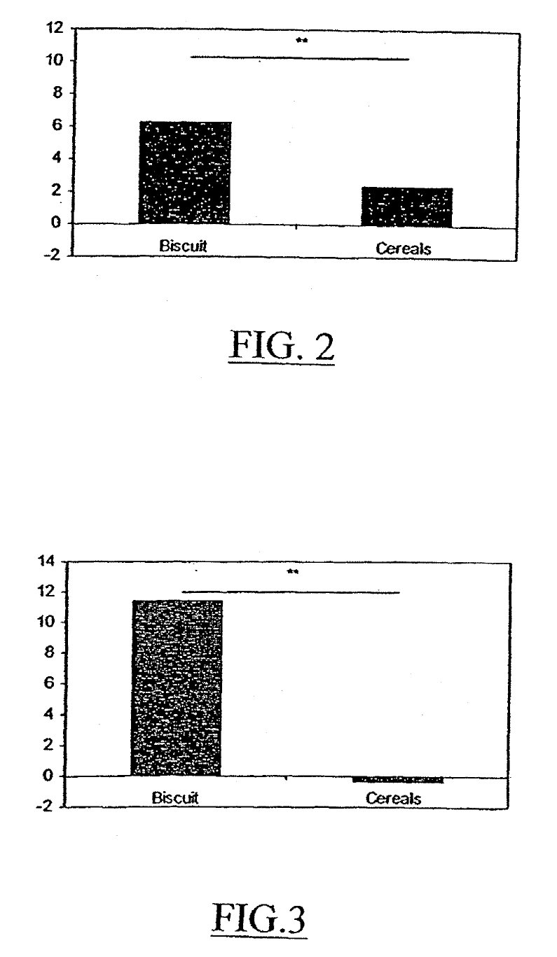

FIG. 2 represents the discrimination between the active lever and the inactive lever 2 hours and 15 minutes after the consumption of breakfast and after 20 minutes of learning (day 1 after familiarization) as a function of the type of breakfast consumed.

FIG. 3 represents the discrimination between the active lever and the inactive lever 2 hours and 15 minutes after the consumption of breakfast and after 20 minutes of learning (day 21 of the familiarization) as a function of the type of breakfast consumed.

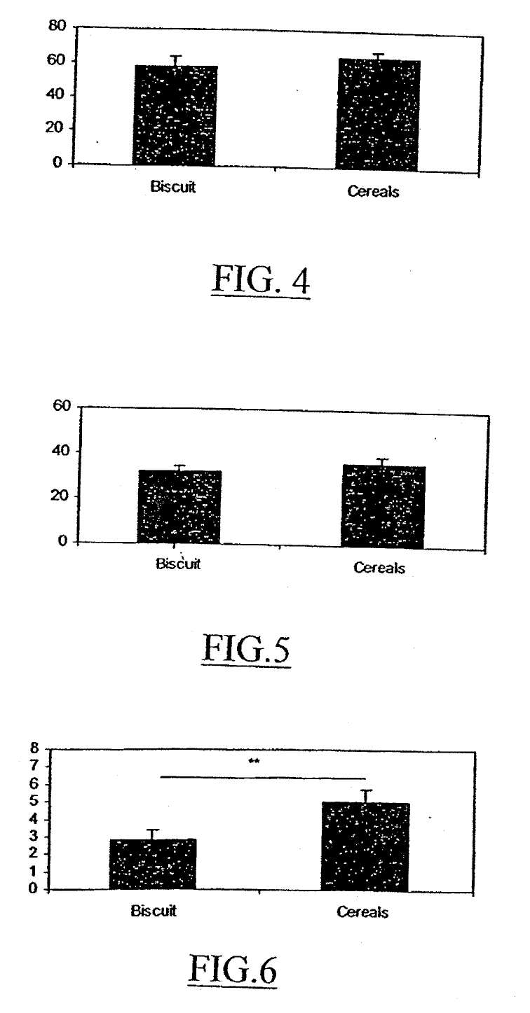

FIG. 4 represents the number of peripheral compartments covered during the 3 minutes of the test and 2 hours and 15 minutes after the consumption of breakfast on day 1 of the familiarization as a function of the type of breakfast consumed.

FIG. 5 represents the number of rightings during the 3 minutes of the test and 2 hours and 15 minutes after the consumption of breakfast on day 1 of the familiarization as a function of the type of breakfast consumed.

FIG. 6 represents the number of entries into the central compartment during the 3 minutes of the test and 2 hours and 15 minutes after the consumption of breakfast on day 1 of the familiarization as a function of the type of breakfast consumed.

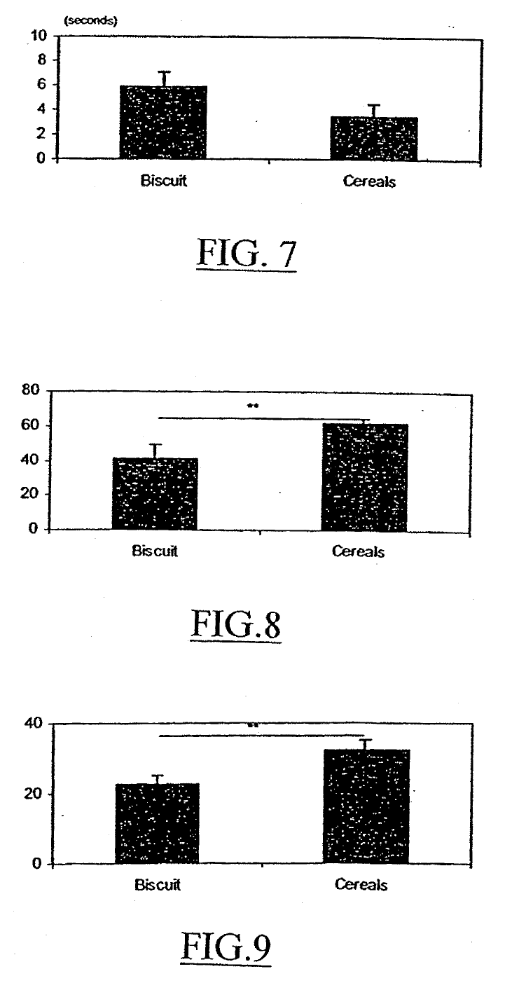

FIG. 7 represents the duration of immobility during the 3 minutes of the test and 2 hours and 15 minutes after the consumption of breakfast on day 1 of the familiarization as a function of the type of breakfast consumed.

FIG. 8 represents the number of peripheral compartments covered during the 3 minutes of the test and 2 hours and 15 minutes after the consumption of breakfast on day 21 of the familiarization as a function of the type of breakfast consumed.

FIG. 9 represents the number of rightings during the 3 minutes of the test and 2 hours and 15 minutes after the consumption of breakfast on day 21 of the familiarization as a function of the type of breakfast consumed.

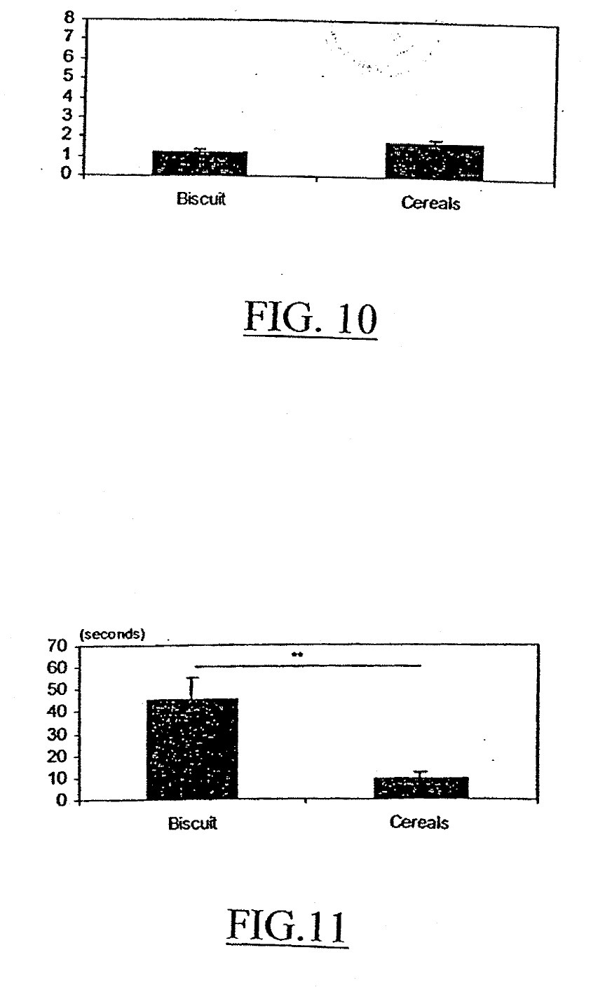

FIG. 10 represents the number of entries into the central compartment during the 3 minutes of the test and 2 hours and 15 minutes after the consumption of breakfast on day 21 of the familiarization as a function of the type of breakfast consumed.

FIG. 11 represents the duration of immobility during the 3 minutes of the test and 2 hours and 15 minutes after the consumption of breakfast on day 21 of the familiarization as a function of the type of breakfast consumed.

### EXAMPLE 1

### Study on the Cognitive Performance of Cereal Products According to the Invention

To examine the effects of the cereal products which can be used according to the invention on cognitive performance, learning within the hours following the consumption of a breakfast composed either of biscuits according to the present invention, or of commercial ready-to-eat cereals, was evaluated.

The nutritional composition of the two products is relatively comparable as attested by table 2. The contents of Mg and Vit C, nutrients which may be involved in the tonus of animals, are also comparable.

TABLE 2
Nutritional composition of the two types of breakfast

| | Biscuit | Cereals |
|---|---|---|
| Water in g/100 g | 3.6 | 2.9 |
| Carbohydrates in g/100 g | 63.5 | 66.7 |
| Starch in g/100 g | 34.0 | 31.4 |
| Sugars in g/100 g | 29.5 | 35.3 |
| Lipids in g/100 g | 17.7 | 20.0 |
| Proteins in g/100 g | 6.5 | 6.5 |
| Vitamin C in mg/100 g | 50.0 | 49.6 |
| Magnesium in mg/100 g | 53.0 | 50.0 |

These two cereal foods are manufactured from similar ingredients (flour, sugar, fat and the like) in similar proportions.

The bioavailability of the starch in the two foods was measured using the Englyst method. The results of these measurements are presented in the following table 3.

TABLE 3
Bioavailability in vitro (Englyst method) of
the carbohydrates of the two types of breakfast

| | | Slowly available |
|---|---|---|
| | Slowly digestible | glucose/total |
| | starch/total starch | available |
| | (%) | carbohydrates (%) |
| Cereals | 2 | 1 |
| Biscuit | 38 | 23 |

Two groups of 24 rats were habituated to consuming a breakfast representing 25% of their energy needs and composed of one of the two products, followed by a period of fasting of 2 hours 30 minutes. This fasting is followed by a free access to food allowing the animals to consume food during the rest of the day. This made it possible to reproduce the consumption habits practiced by people. After about ten days of familiarization, the rats were subjected to learning tests and to measurements of their locomotive activity, at a time corresponding to 2 hours 15 minutes after their breakfast. This critical period is often described as corresponding to times of reduction in attention and “feeling drained”.

The learning test consists in placing the animals in an illuminated room having two levers (one, which is active, switches off the light; the other, which is inactive, is without effect). After pressing on the active lever, the light is switched off for 30 seconds, then switches on again. The rat, spontaneously preferring to be in darkness, therefore gradually learns, by pressing more often on the active lever than on the inactive lever. For the measurement of the locomotive activity, the animal is placed for 3 minutes in an organized room, comprising on the floor a subdivision into 9 compartments, which make it possible to quantify its locomotive activities on the basis of several criteria:

number of peripheral compartments covered

number of rightings

number of entries into the central compartment

duration of immobility (variable deduced from the preceding 3).

Thus, the learning and the locomotive activity were able to be evaluated in the period which follows the consumption of a breakfast composed of two types of carbohydrate foods: breakfast cereals and biscuits. The aim is to check that the biscuit, a little-known carbohydrate food, leads to the same results as breakfast cereals, a well-known carbohydrate food. This comparison was made in the acute phase (on day 1 after the familiarization) and after 3 weeks of “breakfast” regime with one of the two products (on day 21 after the familiarization).

The results were very surprising because the rats which consumed biscuits exhibited learning results which were significantly superior to those of the rats which consumed ready-to-eat cereals. FIGS. 1 and 2 illustrate the very significant differences observed between the two types of condition, both on day 1 and after 3 weeks of regime (D21). The consumption of biscuit is followed by learning which is significantly superior to that following the consumption of cereals.

In parallel, the results obtained for the locomotive activity were also surprising since a very significant difference exists between the two products (FIGS. 3 to 10).

The rats which consumed a biscuit-based breakfast were more calm, whereas the rats which consumed a breakfast based on ready-to-eat cereals were more active and show signs of distress (more passages in the central compartment, this indicating higher distress since the behavior of crossing a room along the diagonal rather than along the walls is unusual in rats).

It is obvious that only the bioavailability of starch makes it possible to explain these differences in results.

Biscuits have significantly more of slowly digestible starch and slowly available glucose than breakfast cereals, which explains their positive action on mental well-being and functioning expressed through improvements in learning. In parallel, breakfast cereals, which are rapidly digested, are thought to rapidly induce a disturbing hunger, increasing the activity and the distress of the animals (in correspondence with the search for food), are thought to reduce their attention with, as a consequence, lesser results of learning. The difficulties of covering the needs of the brain in substrates after ingestion of rapidly digested cereals could contribute to the lesser learning observed.

The biscuits used in this example have, in addition, a glycemic index of 48+/−6.

### EXAMPLE 2

### Comparison of the Effect of Two Cereal Products on the Acquisition of Learning

The short-term effects of the ingestion of two cereal products with equivalent glycemic index and different slowly digestible starch contents are tested in an aversive light stimulus avoidance conditioning test (ALSAT) in adult male Wistar rats.

The products are, on the one hand, biscuits according to the present invention with a low fat content and a glycemic index of 83.6, and, on the other hand, commercial ready-to-eat cereals with a glycemic index of 81.6, the compositions of which are presented in table 4.

TABLE 4
Respective composition of the two types of breakfast
Analyses in vitro

| | Water | Total | Total | Total | | | SDS/TS* | Glycemic |
|---|---|---|---|---|---|---|---|---|
| Products | (w.b) | sugars** | starch | carbohydrates*** | Lipids | Proteins | (%) | index |
| Cereals | 2.8 | 42.7 | 38.0 | 80.7 | 4.7 | 5-7 | 2 | 83.6 ± 8.8 |
| Biscuit | 3.0 | 24.5 | 53.3 | 77.8 | 5.0 | 5-7 | 29 | 81.6 ± 19.4 |

*slowly digestible starch/total starch
**combination of simple sugars (glucose, fructose, sucrose and the like)
***= total sugars + total starch

Twenty-four male Wistar rats of 250 to 340 g in weight are used. The animals are randomly divided into two groups of 12 rats. The rats of each group are marked and grouped in four per cage. The animals are kept in an air-conditioned animal house, at a temperature of 22 to 24° C., and subjected to a 12-hour cycle of light-darkness (light from 11 p.m. to 11 a.m.).

The two groups of rats respectively consume a breakfast composed of biscuits according to the present invention and cereals.

The products used are balanced with respect to the supply of carbohydrates.

The rats of each group are habituated to the products for 4 days (D-3 to D0). For that, small quantities of the products to be tested are introduced into troughs in order to familiarize each of the groups of rats with one of the two products to be tested.

During a period of 11 days (D1 to D11), the products are given to the rats every other day during breakfast (D1, D3, D5, D7, D9 and D11), alternately with the same calorie ration based on dry food (D2, D4, D6, D8 and D10). This period makes it possible to habituate the rats to a dietary rhythm mimicking the period of transient fasting between breakfast and lunch in people, that is to say that the animals are subjected to a postprandial fast period of 150 minutes after the end of breakfast. The breakfast which starts at T0, lasts for a maximum of 30 minutes (T30) and represents 20% of the daily energy supplies for the animals (expressed in Kcal).

Between day 1 and day 10, the animals are subjected to 150 minutes of fasting after the end of breakfast (T180) after which the remainder of the food is given in the form of dry food for the rest of the day (breakfast rhythm). On D11, following the period of fasting (T180), the rats are subjected to the learning test lasting for 20 minutes.

Experimental Device:

The experimental device consists of an isolated cage (50×40×37 cm), strongly illuminated and containing two levers: one which is active, making it possible, when it is operated, to obtain 30 seconds of darkness followed by the return of light, whereas the other lever is inactive (does not cause darkness). Pressing on the active lever during the period of darkness does not provide additional periods of darkness. The rat is placed in the cage for 20 minutes and the number of pressings on each lever is counted during the experiment.

The battery of test, composed of 4 conditioning devices, is fully automated and computer controlled. Thus, no experimenter is present in the room during the test.

This test is carried out on day 11, 180 minutes after the start of breakfast, in order to evaluate the effect of the products on the acquisition of learning in the ALSAT device for 20 minutes.

Variables Recorded:

number of pressings on the active (AL) and inactive (IL) levers,

level of efficiency of the pressings [(AL/AL+IL)×100].

The Mann-Whitney test was used to compare the performance of the rats of the groups for biscuits according to the present invention and for cereals. The Wilcoxon test served to evaluate the discrimination by comparing the active pressings to the inactive pressings of each of the two groups of rats.

The data are expressed as median values and interquartile ranges. The risk threshold is set at 5%. The statistical treatments were carried out using the Statview 4.1 software (Abacus Concept).

Results:

1) Effects of the Products on the total number of pressings on the two levers.

The results obtained are assembled in table 5.

TABLE 5
Total number of pressings during the test
(median values and interquartile ranges)

| | Biscuit | Cereals |
|---|---|---|
| Products | (n = 12) | (n = 12) |
| Total number of pressings | 38 | 39 |
| Median | (32.0-42.5) | (20.0-46) |
| (Qi-Qs) | | |

| | |
|---|---|
| Mann-Whitney test | U = 69; N.S. |

The Mann-Whitney tests do not show significant differences between the rats of the biscuit and cereal groups.

2) Effects of the products on the discrimination between the two active and inactive levers:

The results obtained are assembled in table 6.

TABLE 6
Discrimination between the active and inactive levers
during the test (median values and interquartile ranges)

| | 0-5 | | | |
|---|---|---|---|---|
| Time Products | minutes | 0-10 minutes | 0-15 minutes | 0-20 minutes |
| Biscuit | | | | |
| (n = 12) | | | | |
| AL | 4.5 (2-6) | 9.5 (5.5-14.5) | 16.5 (13.5-20.5) | 21.5 (17.5-30.25) |
| IL | 2.5 (2-6) | 8.5 (4.5-11) | 11.5 (9-15.5) | 15 (12.5-19.5) |
| Wilcoxon test | z = 1.49; | z = 1.81; N.S. | z = 2.71; | z = 2.83; |
| | N.S. | | p < 0.01 | p < 0.005 |
| Cereals | | | | |
| (n = 12) | | | | |
| AL | 4.5 (2.5-7) | 8.5 (6.5-13.5) | 11.5 (10-18) | 20 (12-24) |
| IL | 4 (2.5-7) | 10 (3.5-13) | 11.5 (6.5-17.5) | 17 (8.5-23.5) |
| Wilcoxon test | Z = 0.09; | Z = 1.03; N.S. | Z = 1.20; N.S. | Z = 1.61; N.S. |
| | N.S. | | | |

Surprisingly, it is observed that the rats of the biscuit group significantly discriminate between the active lever and the inactive lever at 15 and 20 minutes of test.

On the other hand, the rats of the cereal group do not discriminate between the two levers throughout the test.

3) Effects of the products on the level of efficiency of the pressings on the active lever:

The results are assembled in table 7.

TABLE 7
Level of efficiency of the pressings during the test
(AL/AL + IL) × 100 (median values and interquartile ranges)

| Products | 0-10 minutes | 0-20 minutes |
|---|---|---|
| Biscuit | 61.4 | 62.4 |
| (n = 12) | (48.2-63.4) | (57.9-64.5) |
| Cereals | 56.3 | 56.1 |
| (n = 12) | (45.1-64.2) | (52.5-59.6) |
| Mann-Whitney test | U = 58.5; N.S. | U = 39; p < 0.06 |

The level of efficiency of the pressings of the rats of the biscuit and cereal groups are not significantly different from each other between 0 and 10 minutes of test.

On the other hand, surprisingly, it is observed that the rats of the biscuit group tend to appear respectively more effective than those of the cereal group over the entire test.

Conclusion:

In the aversive light stimulus avoidance conditioning situation, the total number of pressings on the active and inactive levers is not significantly different between the rats of the biscuit and cereal groups.

However, the rats of the biscuit group significantly discriminate between the active lever and the inactive lever at 15 and 20 minutes of test, whereas those of the cereal group show deficiencies at this level throughout the test.

The differences observed between the rats of the biscuit and cereal groups should be attributed to the quality of the carbohydrates contained in their respective breakfasts. Indeed, these two breakfasts have an equivalent glycemic index and a low fat content but different slowly digestible starch contents. Thus, the beneficial effects observed because of the consumption of a breakfast composed of biscuits according to the present invention, that is to say good learning performance, could result from a better biological and psychological balance.

### EXAMPLE 3

### Comparison of the Effect of Two Cereal Products on the Acquisition of Learning Following a Physical Exhaustion Test

Twenty-four male Wistar rats weighing from 360 to 450 g are used. The rats are marked and divided into groups of four in cages. The animals are kept in an airconditioned animal house, at a temperature of 22-24° C. and are subjected to a 12-hour cycle of light-darkness.

The products tested are biscuits according to the present invention and commercial ready-to-eat cereals.

The nutritional composition of the two products is relatively comparable as shown in the following table 8:

TABLE 8
Nutritional composition of the two types of breakfast

| Products | Biscuit | Cereals |
|---|---|---|
| Carbohydrates (g/100 g) | 63.5 | 71.5 |
| Lipids (g/100 g) | 17.7 | 14.2 |
| Proteins (g/100 g) | 6.5 | 6.9 |
| Energy supply (Kcal/100 g) | 439.3 | 441.4 |

The contents of Mg and Vit C, nutrients which may be involved in the tonus of animals, are also comparable.

These two cereal foods are manufactured from similar ingredients (flour, sugar, fat and the like) in similar proportions.

The bioavailability of the starch in both foods was measured using the Englyst method. The results of these measurements are presented in table 3 of example 1.

After a habituation of one week to the laboratory conditions, the cages of rats are randomly divided into 2 groups: biscuit and cereals (n=24 rats per group), that is six cages per group.

During the 4 days following the habituation period, the small quantities of the products to be tested are respectively introduced into the troughs in order to familiarize the rats of both groups with the new foods.

Both products are given to the rats every other day during breakfast, for a period of 10 days, alternately with the same calorie ration based on dry food. The rations are prepared and then distributed to the animals so that they have equal calorie levels and equal carbohydrate levels between the groups of rats for the biscuits and the cereals. The breakfast, lasting for 30 minutes, represents 20% of the quantity of food consumed daily (a rat consumes daily on average 21 Kcal/100 g of body weight). Following breakfast, the animals are fasted for 150 minutes, after which the remainder of the food is delivered in the form of dry food for the rest of the day (breakfast rhythm).

Physical Exhaustion of the Rats by Forced Swimming:

The day of the test (D10), 40 minutes before the end of the fasting period, 12 rats of each of the two groups are subjected to physical exhaustion by forced swimming. The rats of each of the cages are deposited in four basins (diameter 30 cm, height: 36 cm), filled with water to a level of 22 cm. After 10 minutes of forced swimming, the rats are removed from the basin, carefully dried and returned to their cage before being tested, 30 minutes afterward, in the aversive light stimulus avoidance conditioning test.

Aversive Light Stimulus Avoidance Conditioning Test:

The experimental device is identical to that used in examples 1 and 2.

This test is carried out on day 10, 180 minutes after the start of breakfast, in order to evaluate the effect of the products and of exhaustion on the acquisition of learning in the aversive light stimulus avoidance conditioning test for 20 minutes.

Variables recorded: number of pressings on the active and inactive levers.

Statistical analyses: One factor variance analysis is used to demonstrate a possible heterogeneity in the manipulatory activity of the levers of the rats of the different groups. Where appropriate, it is followed by an unpaired t test to compare the groups of rats in pairs. The paired t test is used to compare the pressings on the active lever and the pressings on the inactive lever by the rats of each group (study of the discrimination). The statistical treatments are carried out using the Statview 4.1 software (Abacus Concept).

Result:

1) Effect of the products on the total number of pressings on both levers:

The results are assembled in table 9.

TABLE 9
Effects of the products on the total number of
pressings (mean values ± SEM)

| | Biscuit | Cereals |
|---|---|---|
| GROUP | (n = 12) | (n = 12) |
| GROUP 1 | 23.33 ± 5.40 | 15.25 ± 2.28 |
| (without physical | | |
| exhaustion) | | |
| GROUP 2 | 14.58 ± 2.61 | 13.92 ± 2.90 |
| (with physical | | |
| exhaustion) | | |

ANOVA does not show heterogeneity in the total number of pressings by the rats of the different biscuit and cereal groups, with or without physical exhaustion.

2) Effect of the products on the discrimination between both active and inactive levers:

To integrate the function of each of the levers, the rats must have pressed on the active lever and on the inactive lever. In order to properly estimate the discrimination between active lever and inactive lever, the rats which did not press on either of the two levers are removed from the study.

a) Discrimination between the levers during the first 10 minutes of test:

The results are assembled in tables 10 and 11.

TABLE 10
Effects of the products on the discrimination
between the levers during the first 10 minutes of test,
without physical exhaustion (mean values ± SEM)

| | | Biscuit | Cereals |
|---|---|---|---|
| GROUP | LEVER | (n = 10) | (n = 10) |
| Without | AL | 7.90 ± 1.97 | 6.00 ± 0.70 |
| physical exhaustion | IL | 5.20 ± 1.25 | 4.50 ± 0.58 |
| Paired t test | | t = 2.49; p < 0.05 | t = 1.50; N.S. |
| (AL VS IL) | | | |

TABLE 11
Effects of the products on the discrimination between
the levers during the first 10 minutes of test,
with physical exhaustion (mean values ± SEM)

| | | Biscuit | Cereals |
|---|---|---|---|
| GROUP | LEVER | (n = 10) | (n = 9) |
| With physical exhaustion | AL | 4.90 ± 1.06 | 4.00 ± 0.70 |
| | IL | 4.20 ± 0.89 | 4.67 ± 0.81 |

| | | |
|---|---|---|
| Paired t test (AL VS IL) | t = 0.86; N.S. | t = 1.21; N.S. |

During the first 10 minutes of learning, only the rats of the biscuit group which have not been subjected to physical exhaustion significantly discriminate between both levers.

b) Discrimination between the levers during the 20 minutes of test:

The results are assembled in tables 12 and 13.

TABLE 12
Effects of the products on the discrimination
between the levers during the 20 minutes of test,
without physical exhaustion (mean values ± SEM)

| | | Biscuit | Cereals |
|---|---|---|---|
| GROUP | LEVER | (n = 11) | (n = 11) |
| Without | AL | 16.73 ± 4.09 | 9.27 ± 1.48 |
| physical exhaustion | IL | 8.64 ± 1.76 | 7.36 ± 0.86 |

| | | |
|---|---|---|
| Paired t test (AL vs IL) | t = 2.62; p < 0.05 | t = 1.38; N.S. |

TABLE 13
Effects of the products on the discrimination
between the levers during the 20 minutes of test, with
physical exhaustion (mean values ± SEM)

| | | Biscuit | Cereals |
|---|---|---|---|
| GROUP | LEVER | (n = 10) | (n = 11) |
| With physical exhaustion | AL | 10.30 ± 1.46 | 7.64 ± 1.74 |
| | IL | 7.10 ± 0.95 | 7.18 ± 1.39 |

| | | |
|---|---|---|
| Paired t test (AL vs IL) | t = 2.71; p < 0.05 | t = 0.51; N.S. |

Surprisingly, it is observed that during the 20 minutes of learning, the rats of the biscuit group with or without physical exhaustion significantly discriminate between both levers. This is not the case for the rats of the cereal groups.

Conclusion:

The overall pressing activity in the Aversive Light Stimulus Avoidance Conditioning test is statistically equivalent between the rats of the biscuit and cereal groups, whether they have been subjected or otherwise to the physical exhaustion test in the form of forced swimming.

The rats of the biscuit group, which have not been subjected to physical exhaustion, show good learning performance at the end of the first 10 minutes of test.

Those of the biscuit group, which have been subjected to the physical exhaustion test and those of the two cereal groups, tested under the same conditions, do not show discrimination between the two levers.

At the end of 20 minutes of test, the rats of the biscuit groups show good performance in the aversive light stimulus avoidance test even after a physical exhaustion test, whereas those of the cereal groups show a learning deficiency (with or without physical exhaustion).

The retardation in acquiring discrimination between the levers by the rats of the biscuit group which were subjected to physical exhaustion is thought to be linked to the intensity of the forced swimming test which would have exhausted part of their available energy.

The differences observed between the rats of the biscuit and cereal groups can only be attributed to the quality of the carbohydrates contained in their respective breakfasts. Indeed, the rats of the biscuit group which consumed more slowly digestible starch and slowly available glucose during breakfast have sufficient energy reserves to withstand the forced fasting and the physical exhaustion test and to thus achieve good learning performance in the Aversive Light Stimulus Avoidance Conditioning test.

Examples of Cereal Products Which can be Used According to the Invention

The following examples of biscuits are given in the table below as a guide and without limitation.

| | LU Petit | | | | LU Petit | |
|---|---|---|---|---|---|---|
| | Déjeuner ® Honey | | Prince petit | | Déjeuner ® | |
| | Chocolate | | déjeuner ® | | Chocolate | |
| | | % | | % | | % |
| | g/100 g | total | g/100 g | total | g/100 g | total |
| Version | of dry matter | energy | of dry matter | energy | of dry matter | energy |
| Proteins | 7 | 6 | 6.5 | 6 | 7 | 6 |
| Lipids | 17 | 34 | 18 | 35 | 17 | 34 |
| Fiber | 4 | | 4.5 | | 6 | |
| Carbohydrates | 68 | 60 | 68 | 59 | 66 | 60 |
| Sugars | 31 | | 27.8 | | 31 | |
| Starch | 37 | | 40.2 | | 35 | |
| Vitamins | 25% RDI | | 30% RDI | | 25% RDI | |
| (B1, B2, | | | | | | |
| PP, B6, | | | | | | |
| B9, B12, | | | | | | |
| B5) | | | | | | |
| Calcium | 25% RDI | | 30% RDI | | 25% RDI | |
| Iron | 25% RDI | | 30% RDI | | 25% RDI | |
| Magnesium | 15% RDI | | | | 15% RDI | |
| Energy | 454 | | 460 | | 445 | |

The following compositions of sweet biscuits which can be used according to the invention are given as a guide and without limitation.

| Composition in % by weight | Forming technology | | |
|---|---|---|---|
| relative to the dough | Laminated | Rotary | Wire cut |
| Flour | 52 to 64 | 40 to 63 | 28 to 40 |
| Sugar | 13 to 22 | 12 to 33 | 14 to 22 |
| Glucose syrup | 0 to 4 | 0 to 4 | 0 to 2 |
| Salt | 0.2 to 1 | 0.2 to 1 | 0.2 to 0.6 |
| Fat | 3 to 16 | 5 to 22 | 14 to 20 |
| Water | 10 to 20 | 1 to 8 | 3 to 6 |
| Leavening agent | 0.1 to 2 | 0 to 0.6 | 0.9 to 1.5 |
| Emulsifier | 1.5 to 4 | 0 to 2 | 0 to 0.5 |
| Powdered milk derivatives | 0 to 2 | 0 to 2 | 0 to 2 |
| Powdered egg | 0 to 2 | 0 to 2 | 2 to 4 |
| Inclusions (chocolate, | | 0 to 15 | 10 to 20 |
| nougatine, fruit) | | | |
| Cocoa powder | 0 to 8 | 0 to 8 | 0 to 30 |
| Recycled ground biscuits | 0 to 5 | 0 to 10 | 0 to 5 |

The following compositions of crackers which can be used according to the invention are given as a guide and without limitation.

| | Enzymatic or chemical | Fermented |
|---|---|---|
| % by weight relative to the dough | cracker | cracker |
| Flour | 50 to 0 | 65 to 75 |
| Sugar | 0 to 10 | 0 to 0.2 |
| Glucose syrup | 0.5 | 0 to 2 |
| Salt | 0.1 to 2 | 0.5 to 2 |
| Fat | 5 to 15 | 5 to 12 |
| Water | 10 to 20 | 13 to 20 |
| Leavening agent or enzyme | 0.5 to 2 | 0.1 to 0.2 |
| Biological yeast | 0 | 0 to 0.5 |
| Powdered milk derivatives | 0 to 4 | 0 to 5 |
| Malt | 0 to 5 | 0 to 8 |
| Ground biscuits | 0 to 5 | 0 to 5 |

These cracker doughs are then baked, sprayed with fat and flavoring substance (0 to 20%).

1. The use of a cereal product having a slowly digestible starch content relative to the total starch content greater than about 12% by weight, preferably greater than about 20%, to improve the memorization, attention, concentration, vigilance and/or mental well-being in a person, and particularly in a child and an adolescent.

2. The use as claimed in claim 1, characterized in that the slowly available glucose content relative to the total carbohydrate content of the cereal product is greater than about 10%, preferably greater than about 15%.

3. The use as claimed in claim 1 or 2, characterized in that the sugar content of the cereal product is from 2 to 40 g per 100 g of dry matter.

4. The use as claimed in claim 3, characterized in that the sugar is a monosaccharide and/or disaccharide, preferably glucose, sucrose, fructose and/or maltose.

5. The use as claimed in one of the preceding claims, characterized in that the lipid content of the cereal product is from 3 to 25 g per 100 g of dry matter, preferably from 10 to 20 g per 100 g of dry matter.

6. The use as claimed in claim 5, characterized in that the lipid content of the cereal product is from 14 to 20 g per 100 g of dry matter.

7. The use as claimed in one of the preceding claims, characterized in that the starch content of the cereal product is from 30 to 70 g per 100 g of dry matter, preferably from 34 to 60 g per 100 g.

8. The use as claimed in one of the preceding claims, characterized in that the protein content of the cereal product is from 5 to 11 g per 100 g of dry matter.

9. The use as claimed in one of the preceding claims, characterized in that the carbohydrate content of the cereal product is from 60 to 90 g per 100 g of dry matter.

10. The use as claimed in one of the preceding claims, characterized in that the cereal product is consumed at breakfast.

11. The use as claimed in one of the preceding claims, characterized in that the cereal product is of the biscuit or cracker type.
